(19) United States
(12) Patent Application Publication
(10) Pub. No.: US 2017/0253585 A1
(43) Pub. Date: Sep. 7, 2017

(54) PROCESSES FOR THE PREPARATION OF INTERMEDIATES OF RALTEGRAVIR

(71) Applicant: MYLAN LABORATORIES LTD., Hyderabad (IN)

(72) Inventors: Siva Ram Prasad Vellanki, Hyderabad (IN); Raja Babu Balusu, Hyderabad (IN); Kiran Mitchanagatla, Hyderabad (IN)

(21) Appl. No.: 15/116,100

(22) PCT Filed: Feb. 3, 2015
(86) PCT No.: PCT/IB2015/050808

§ 371 Date: Aug. 2, 2016

(30) Foreign Application Priority Data
Feb. 3, 2014 (IN) 471/CHE/2014

(51) Int. Cl. C07D 413/12; C07D 239/557

(52) U.S. Cl. CPC C07D 413/12; C07D 239/557

(57) ABSTRACT

The present disclosure provides a process for the preparation of 2-(2-amino propan-2-yl)-N-(4-fluorobenzyl)-5-hydroxy-1-methyl-6-oxo-1,6-dihydro-pyrimidine-4-carboxamide by debenzylation of benzyl(2-{4-[(4-fluorobenzyl)carbamoyl]-5-hydroxy-1-methyl-6-oxo-1,6-dihydropyrimidin-2-yl}propan-2-yl)carbamate. This process may be used in the synthesis of raltegravir and pharmaceutically acceptable salts thereof.

### CROSS-REFERENCE TO RELATED APPLICATIONS

This application, in its entirety, claims the benefit of earlier Indian provisional patent application No. 471/CHE/2014 filed on Feb. 3, 2014.

### BACKGROUND OF THE INVENTION

Field of the Invention

The present disclosure relates generally to methods of synthesis of pharmaceutical products, and more specifically to a process for the preparation of 2-(2-amino propan-2-yl)-N-(4-fluorobenzyl)-5 -hydroxy-1-methyl-6-oxo-1,6-dihydropyrimidine-4-carboxamide and its further conversion into N-(4-fluorobenzyl)-5-hydroxy-1-methyl-2-(2-{[(5-methyl-1,3,4-oxadiazol-2-yl)carbonyl]amino}-2-propanyl)-6-oxo-1,6-dihydro-4-pyrimidinecarboxamide and pharmaceutically acceptable salts thereof.

Background of the Invention

Raltegravir is an antiretroviral drug, which in its potassium salt form, is marketed under the brand name ISENTRESS® by Merck & Co. It is often used in combination with other anti-retroviral drugs to treat human immunodeficiency virus (HIV) infection. Raltegravir is a first line HIV-integrase strand transfer inhibitor drug that targets integrase, an HIV enzyme that integrates viral genetic material into human chromosomes. Raltegravir potassium is chemically known as 4-[N-(4-fluorobenzyl)carbamoyl}-1-methyl-2-{1-methyl-1-(5-methyl-1,3,4-oxadiazol-2-ylcarboxamido)ethyl}-6-oxo-1,6-dihydropyrimidin-5-olate potassium salt. It has a structure represented below by Formula I.

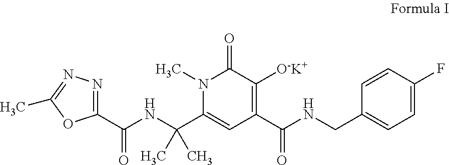

Raltegravir and its pharmaceutically acceptable salts are disclosed in U.S. Pat. No. 7,169,780, which is hereby incorporated by reference.

The process for the preparation of Raltegravir is disclosed in U.S. Pat. No. 7,169,780 and U.S. Patent App. Pub. No. 2010/0280244, which are both hereby incorporated by reference.

There exists a need to provide a cost-effective and simple process for synthesizing raltegravir. The present invention provides an improved and simple process which employs debenzylation of a benzyl-protected intermediate with an acid to produce raltegravir.

### SUMMARY OF THE INVENTION

One aspect of the present disclosure is to provide a process for preparation of 2-(2-amino propan-2-yl)-N-(4-fluorobenzyl)-5-hydroxy-1-methyl-6-oxo-1,6-dihydropyrimidine-4-carboxamide (shown below as Formula III) by debenzylation of benzyl(2-{4-[(4-fluorobenzyl)carbamoyl]-5-hydroxy-1-methyl-6-oxo-1,6-dihydropyrimidin-2-yl}propan-2-yl)carbamate (shown below as Formula IV). Formula III may be formed as an intermediate during the synthesis of raltegravir.

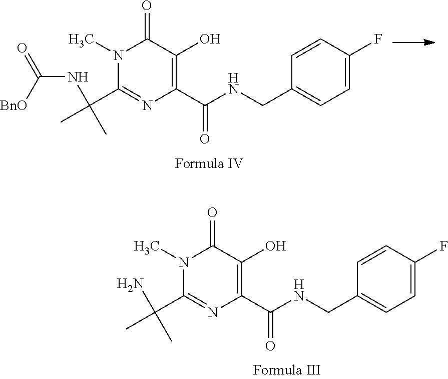 [SMILES: Cn1c(C(C)(C)NC(=O)OCc2ccccc2)nc(C(=O)NCc2ccc(F)cc2)c(O)c1=O ; Cn1c(C(C)(C)N)nc(C(=O)NCc2ccc(F)cc2)c(O)c1=O]

### DETAILED DESCRIPTION OF THE INVENTION

It is to be understood that the description of the present invention has been simplified to illustrate elements that are relevant for a clear understanding of the invention, while eliminating, for purposes of clarity, other elements that may be well known.

The present invention discloses novel methods for producing intermediates in the synthesis of raltegravir.

One aspect of the present disclosure is to provide a process for preparation of 2-(2-amino propan-2-yl)-N-(4-fluorobenzyl)-5-hydroxy-1-methyl-6-oxo-1,6-dihydropyrimidine-4-carboxamide (shown below, and hence forward referred to as as ‘Formula III’) by debenzylation of benzyl (2-{4-[(4-fluorobenzyl)carbamoyl]-5-hydroxy-1-methyl-6-oxo-1,6-dihydropyrimidin-2-yl}propan-2-yl)carbamate (shown below, and hence forward referred to as ‘Formula IV’). Formula III may be formed as an intermediate during the synthesis of raltegravir.

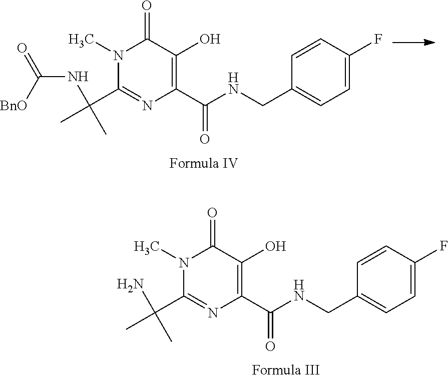 [SMILES: Cn1c(C(C)(C)NC(=O)OCc2ccccc2)nc(C(=O)NCc2ccc(F)cc2)c(O)c1=O ; Cn1c(C(C)(C)N)nc(C(=O)NCc2ccc(F)cc2)c(O)c1=O]

In one embodiment of the present invention, the process for the preparation of Formula III may be carried out by the following steps:
a) debenzylation of Formula IV in the presence of an acid; and
b) isolating Formula III.

According to the present invention, Formula IV may be debenzylated to produce Formula III. This reaction may occur in the presence of an acid, which may be organic or inorganic. Within the context of the present invention, suitable organic acid include, as examples, acetic acid, trifluoroacetic acid, trifluoromethanesulfuric acid, and formic acid. Suitable inorganic acids include, as examples, hydrochloric acid, hydrobromic acid, sulfuric acid, hydrofluoric acid, boric acid, tetrafluoroboric acid, and orthophosphoric acid. In some embodiments of the invention, it has been found that the aqueous form of the inorganic acid is particularly useful for carrying out this step. In particular, aqueous hydrobromic acid was found to be effective.

According to the present disclosure, the debenzylation of Formula IV to give Formula III may be carried out in the presence of a solvent. Within the context of the present invention, suitable solvents include, as examples, acetone, methanol, ethanol, n-propanol, 2-propanol, and mixtures thereof.

According to the present disclosure, the debenzylation of Formula IV may be carried out at a temperature ranging from about 40° C. to about 100° C. It has been found that a temperature range of about 60° C. to about 65° C. is particularly effective for carrying out the debenzylation reaction.

According to the present disclosure, after completion of the debenzylation reaction, the reaction mass may then be cooled to about 5° C. to about 20° C., at which time the pH of the solution may be adjusted to about 7.0 to 12.0, particularly 7.0 to 8.0. The mixture may then be cooled further to about 0-5° C. and stirred to isolate Formula III. In some embodiments of the invention, cooling to a temperature between about 10 and 15° C. prior to adjusting the pH has been found to be particularly effective when carrying out this reaction. After isolation, the product may then be optionally washed with a solvent to enhance purification. Examples of suitable washing solvents include alcohol solvents, as examples, methanol, ethanol, n-propanol and 2-propanol.

In another aspect of the present invention, the obtained Formula III may be further converted into raltegravir or pharmaceutically acceptable salts thereof by processes disclosed in U.S. Pat. No. 7,754,731, PCT Publication Nos. WO2011024192 and WO 2010140156, as well as Indian Patent Application Serial No. 736/CHE/2012, which are hereby incorporated by reference.

With all of the reactions disclosed above, one of skill in the art will recognize that the reaction conditions (e.g., reaction time or temperature) may be adjusted to achieve appropriate yield without undertaking undue experimentation and without departing from the scope of the present disclosure.

Raltegravir or pharmaceutically acceptable salts thereof, prepared by the processes disclosed in the present invention may be incorporated into a pharmaceutical formulation for the treatment of HIV in human patients. Numerous types of pharmaceutical formulations may be employed, including tablets, chewable tablets, and oral suspensions. When formulated as a tablet, the formulation may include such excipients as calcium phosphate dibasic anhydrous, hypromellose 2208, lactose monohydrate, magnesium stearate, microcrystalline cellulose, poloxamer 407 (contains 0.01% butylated hydroxytoluene as antioxidant), sodium stearyl fumarate. In addition, the tablet may include a film coating that may contain the following inactive ingredients: black iron oxide, polyethylene glycol 3350, polyvinyl alcohol, red iron oxide, talc and titanium dioxide. In some embodiments, the raltegravir or pharmaceutically acceptable salts thereof may be included in a chewable tablet. Such formulations may include, as examples of appropriate excipients, ammonium hydroxide, crospovidone, ethylcellulose 20 cP, fructose, hydroxypropyl cellulose, hypromellose 2910/6 cP, magnesium stearate, mannitol, medium chain triglycerides, monoammonium glycyrrhizinate, natural and artificial flavors (orange, banana, and masking that contains aspartame), oleic acid, PEG 400, red iron oxide, saccharin sodium, sodium citrate dihydrate, sodium stearyl fumarate, sorbitol, sucralose and yellow iron oxide. In other embodiments, the pharmaceutical formulation may be an oral suspension. The formulation intended for oral suspension may include excipients such as ammonium hydroxide, artificial flavorings, natural flavorings, carboxymethylcellulose sodium, crospovidone, ethylcellulose 20 cP, fructose, hydroxypropyl cellulose, hypromellose 2910/6 cP, macrogol/PEG 400, magnesium stearate, maltodextrin, mannitol, medium chain triglycerides, microcrystalline cellulose, monoammonium glycyrrhizinate, oleic acid, sorbitol, sucralose, and sucrose.

In treatment of patients with HIV, raltegravir or pharmaceutically acceptable salts thereof, prepared by the processes disclosed in the present invention may also be administered in conjunction with other active pharmaceutical ingredients, including efavirenz, fosamprenavir, ritonavir, tipranavir, rifampin, tenofovir, lamivudine, and emtricitabine.

In view of the above description and the examples below, one of ordinary skill in the art will be able to practice the invention as claimed without undue experimentation. The foregoing will be better understood with reference to the following examples that detail certain procedures for the preparation of molecules, compositions and formulations according to the present invention. All references made to these examples are for the purposes of illustration. The following examples should not be considered exhaustive, but merely illustrative of only a few of the many aspects and embodiments contemplated by the present disclosure.

### Example 1

### Preparation of Formula III

A mass of 730 g of aqueous hydrobromic acid was added to 100 g of benzyl (2-{4-[(4-fluorobenzyl)carbamoyl]-5-hydroxy-1-methyl-6-oxo-1,6-dihydropyrimidin-2-yl}propan-2-yl)-carbamate at 28±3° C. and stirred for 30 minutes. The reaction mixture temperature was raised to 62±3° C. and stirring continued for 2 hours. After completion of the reaction, the reaction mixture was cooled to 28±3° C. and 300 mL of purified water was added. The reaction mixture was stirred and cooled to 12±3° C. The pH of the reaction mixture was adjusted to 7.0-8.0 with a sodium hydroxide solution and further cooled to 3±2° C. The reaction mixture was stirred for about 3-4 hrs. The product was filtered then washed with water. Methanol (500 mL) was added to the wet material at 28±3° C. and stirred for 1 hour to obtain a solid. The solid was filtered then washed with methanol. The product was dried under vacuum at 55±3° C. to get 2-(2-amino propan-2-yl)-N-(4-fluorobenzyl)-5-hydroxy-1-methyl-6-oxo-1,6-dihydropyrimidine-4-carboxamide (Formula III).

1. A process for the preparation of 2-(2-amino propan-2-yl)-N-(4-fluorobenzyl)-5-hydroxy-1-methyl-6-oxo-1,6-dihydropyrimidine-4-carboxamide of formula III, comprising:
a. debenzylation of benzyl(2-{4-[(4-fluorobenzyl)carbamoyl]-5-hydroxy-1-methyl-6-oxo-1,6-dihydropyrimidin-2-yl}propan-2-yl)carbamate (Formula IV) in the presence of an acid; and
b. isolating 2-(2-amino propan-2-yl)-N-(4-fluorobenzyl)-5-hydroxy-1-methyl-6-oxo-1,6-dihydropyrimidine-4-carboxamide (Formula

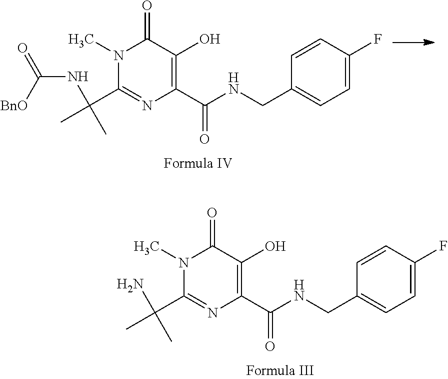 [SMILES: Cn1c(C(C)(C)NC(=O)OCc2ccccc2)nc(C(=O)NCc2ccc(F)cc2)c(O)c1=O ; Cn1c(C(C)(C)N)nc(C(=O)NCc2ccc(F)cc2)c(O)c1=O]

2. The process according to claim 1, wherein the acid is an organic acid or an inorganic acid.

3. The process according to claim 2, wherein organic acid is selected from the group consisting of acetic acid, trifluoroacetic acid, trifluoromethanesulfuric acid, and formic acid.

4. The process according to claim 2, wherein the inorganic acid is selected from the group consisting of hydrochloric acid, hydrobromic acid, sulfuric acid, hydrofluoric acid, boric acid, tetrafluoroboric acid, and orthophosphoric acid.

5. The process according to claim 2, wherein the inorganic acid is aqueous hydrobromic acid.

6. The process according to claim 1, wherein debenzylation of compound of Formula IV is carried out at a temperature between about 40° C. and about 100° C.

7. The process according to claim 1, wherein the isolating step comprises cooling the reaction mixture and adjusting pH of the reaction mixture to between about 7 and about 12 to form a solid comprising 2-(2-amino propan-2-yl)-N-(4-fluorobenzyl)-5-hydroxy-1-methyl-6-oxo-1,6-dihydropyrimidine-4-carboxamide (Formula III).

8. The process according to claim 7, wherein the solid is filtered and washed with water.

9. The process according to claim 7, further comprising the step of purifying 2-(2-amino propan-2-yl)-N-(4-fluorobenzyl)-5-hydroxy-1-methyl-6-oxo-1,6-dihydropyrimidine-4-carboxamide (Formula III) by washing said solid with an alcohol.

10. A process for the preparation of raltegravir, the process comprising the step of converting 2-(2-amino propan-2-yl)-N-(4-fluorobenzyl)-5-hydroxy-1-methyl-6-oxo-1,6-dihydropyrimidine-4-carboxamide of Formula III 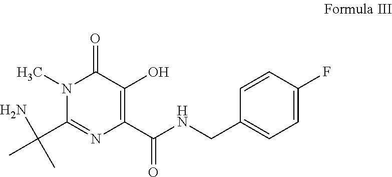 [SMILES: Cn1c(C(C)(C)N)nc(C(=O)NCc2ccc(F)cc2)c(O)c1=O]
as obtained by the process of claim 1 to raltegravir or a pharmaceutically acceptable salt thereof.
